(19) United States
(12) Patent Application Publication
(10) Pub. No.: US 2012/0173249 A1
(43) Pub. Date: Jul. 5, 2012

(54) Absorbent Article With Integrated Machine Readable Code

(75) Inventors: Robert Lee Popp, Greenville, WI (US); Andrew Mark Long, Appleton, WI (US); Thomas Michael Ales, Neenah, WI (US); Kristina Smits, Menasha, WI (US)

(73) Assignee: Kimberly-Clark Worldwide, Inc., Neenah, WI (US)

(21) Appl. No.: 12/982,174

(22) Filed: Dec. 30, 2010

(51) Int. Cl. A61F 13/84; G06Q 30/00; G06K 19/06; G06K 19/00; A61F 13/49; A61F 13/60

(52) U.S. Cl. 705/1.1; 604/385.01; 604/385.3; 206/494; 604/385.05; 235/494; 235/487

(57) ABSTRACT

Absorbent articles are disclosed that include a machine readable code. The machine readable code, once scanned by a suitable reader device, provides access to information related to the absorbent article. The information may relate to use or disposal of the article. In other embodiments, the information may comprise promotional materials, such as coupons, rebates and news about related products, new products, or the like. The present disclosure is particularly directed to applying and positioning the machine readable codes on the absorbent articles so that the codes are noticeable and can be scanned.

### BACKGROUND

Absorbent articles such as diapers, training pants, incontinence products, feminine hygiene products, and the like conventionally include a liquid permeable bodyside liner, a liquid impermeable outer cover, and an absorbent core. The absorbent core is typically located inbetween the outer cover and the liner for taking in and retaining body fluids exuded by the wearer, such as urine.

The absorbent core can be made of, for instance, superabsorbent particles. Many absorbent articles, especially those sold under the trade name HUGGIES™ by the Kimberly-Clark Corporation, are incredibly efficient at absorbing liquids and other body fluids. Absorbent articles today are also easier to don, are very comfortable to wear, prevent against leakage, and also provide or possess various other features and advantages not available previously.

Recently, those skilled in the art have suggested making absorbent articles interactive with the user and the caregiver. In particular, those skilled in the art have proposed incorporating features into the absorbent articles that convey information to the wearer and/or the caregiver. For instance, various different types of wetness sensing devices have been proposed for incorporation into absorbent articles. Wetness indicators may include alarm devices that are designed to assist parents or attendants identify a wet condition early on. The devices are designed to produce either a visible or an audible signal.

In U.S. Patent Application Publication No. 2009/0155753, which is incorporated by reference, a system for tracking behavior is described that can be used in conjunction with an absorbent article. In particular, one or more sensors may be incorporated into an absorbent article that provide information or data into a computing device for tracking the behavior of children or those wearing the absorbent article.

In U.S. Patent Application Publication No. 2009/0326491, which is also incorporated herein by reference, a method is disclosed for providing feedback to a caregiver regarding the progress that is being made in toilet training a child. The method, for instance, may be used to determine when to select a next generation product for facilitating toilet training.

The above products and methods have provided great advances in the art. Through the above products and methods, absorbent articles are not only capturing body fluids but are also becoming interactive by providing useful information to the wearer and/or caregiver during use of the product. A need still exists, however, for further advances in the evolution of absorbent articles and for using the absorbent article as a source of information. More particularly, a need exists for a new method of enabling access to information and/or linking information to a user through the use of an absorbent article. The information, for instance, can relate to consumer needs and/or marketing desires. A need also exists for a method of providing access to information through the use of an absorbent article without significantly increasing the cost of the absorbent article.

### SUMMARY

In general, the present disclosure is directed to absorbent articles that include an information conveying and/or information access device that can be easily incorporated into the article and that can provide a user with access to information that is related to the product. In one embodiment, for instance, the present disclosure is directed to incorporating a machine readable code, such as a two dimensional code, into an absorbent article that, when read by a suitable reader device, provides information about the product. The information conveyed by the code, for instance, can be related to the health or another characteristic of the wearer, can provide information about proper use of the product, or can include promotional information for purchasing further units of the product or related products.

In one embodiment, the machine readable code may comprise a graphic that is applied directly to the absorbent article or applied to a label that is attached to the absorbent article. Applying a machine readable code to an absorbent article in accordance with the present disclosure may provide various advantages and benefits. For instance, the machine readable code can be incorporated into the absorbent article without adding any substantial cost to the article.

In one embodiment, for instance, the present disclosure is directed to an absorbent article that includes at least one machine readable code for providing access to information related to the absorbent article. In one embodiment, the absorbent article includes a crotch region positioned in between a front region and a back region. The absorbent article includes an inside surface that faces a wearer when worn and an opposite outside surface. The absorbent article includes a front waist edge and a back waist edge that define a waist opening opposite two leg openings. The absorbent article has a longitudinal length that extends from the front waist edge to the back waist edge along a longitudinal center line. The absorbent article can optionally further include two opposing side panels that connect the front region to the back region above the leg openings. A front elastic waist region may be located adjacent to the waist opening in the front of the article and a back elastic waist region may be located along the waist opening on the back region of the article.

The absorbent article includes an outer cover having an exterior surface and an interior surface. An absorbent structure is positioned adjacent to the interior surface of the outer cover. The absorbent structure includes a front longitudinal end, a back longitudinal end, a first transverse side, and an opposite second transverse side. In one embodiment, the absorbent article may further include a liquid permeable liner. The absorbent structure may be positioned between the outer cover and the liquid permeable liner.

As described above, the absorbent article may further include a machine readable code that is configured to provide information related to the absorbent article when scanned by a suitable reader device. The machine readable code is located on the absorbent article at a location that is capable of being scanned by a suitable reader device. For example, the machine readable code may be positioned on the absorbent article below the waist opening a distance that is at least 10% of the longitudinal length of the absorbent article. The machine readable code may also be positioned on the absorbent article at a location that coincides with being between the first and second transverse sides of the absorbent structure. In particular, the machine readable code is placed on a relatively flat surface that does not include wrinkles, folds, or a significant amount of undulations.

In one embodiment, for instance, a machine readable code may be positioned on an outside surface of the absorbent article. Alternatively, the machine readable code may be scannable from an inside surface of the absorbent article within the location described above.

In one embodiment, the machine readable code is positioned on the absorbent article below the waist opening a distance that is at least 10% of the longitudinal length of the absorbent article, such as at least about 15% of the longitudinal length of the absorbent article. For example, when the absorbent article includes a front waist elastic region and/or a back waist elastic region, the machine readable code may be positioned below the waist elastic regions towards the crotch region in relation to the waist opening.

In certain embodiments, the machine readable code may be placed on the absorbent article at a location that avoids places where the article is known to form folds that appear on the outside surface of the article or on the inside surface of the article. In one embodiment, for instance, the absorbent article may include a first leg elastic that at least partially surrounds the first leg opening and a second leg elastic that at least partially surrounds the second leg opening. The machine readable code may be positioned above a transverse line that is tangential to a longitudinal extent of the leg elastics. In particular, the machine readable code may be positioned in between the longitudinal extent of the leg elastics and a distance below the waist opening that is at least 10% of the longitudinal length of the absorbent article.

In order for the machine readable code to be easily scannable, the machine readable code may also be placed on the absorbent article at regions where the stiffness of the article is increased. For example, in one embodiment, the machine readable code may be positioned on the absorbent article such that it coincides with the position of the absorbent structure within the article. In an alternative embodiment, the absorbent article may include a stiffening agent that increases the stiffness of the outer cover or liquid permeable liner of the absorbent article and that creates a region on the absorbent article for location of the machine readable code. The stiffening agent, for instance, may comprise a coating applied to the absorbent article, such as an adhesive that increases the stiffness. Alternatively, the stiffening agent may comprise a separate piece of material that is attached to the absorbent article and that is registered where the machine readable code is located. The separate piece of material may comprise any material that allows for easy scanning of the code. The separate piece of material, for instance, may comprise a film, a fabric, or any other suitable sheet material.

In one embodiment, the machine readable code may be applied to a label that is then attached to the absorbent article. In one embodiment, for instance, the label may be positioned on the inside surface of the absorbent article in the back region. In this manner, the machine readable code will appear as a typical garment tag on the article.

In one embodiment, the absorbent article may include at least one tab member located on one of the side panels. For instance, in one embodiment, the absorbent article may include a first tab member on the first side panel and a second tab member on the second side panel. The tab members may be functional or non-functional. When functional, for instance, the tab members may attach to the front region of the absorbent article for attaching the front region to the back region. For example, the tab members may include a fastening member such as an adhesive or hook members that attach to the front region. In accordance with the present disclosure, a machine readable code may be applied to at least one of the tab members. In one embodiment, for instance, a first portion of the machine readable code may be present on a first tab member and a second portion of the machine readable code may be present on the second tab member. When the tab members are brought together, the machine readable code is then complete and can be scanned.

In yet another embodiment, the machine readable code may be present on one of the tab members. On the opposing tab members, on the other hand, instructions may be printed for using the code.

In general, the present disclosure is not only directed to placing machine readable codes on absorbent articles but also to accentuating the codes so that consumers become familiar with the codes and are encouraged to interact with them. Thus, in addition to placing the code at a scannable location, in certain embodiments, the code may also be one focal point of the product. For example, in one embodiment, the machine readable code can be placed not only at a prominent location on the product but may also be visible at the point of purchase. For instance, a plurality of absorbent articles may be contained within a package made from a film. The package may include at least one clear zone that shows the machine readable code on the product through the package. The machine readable code can also be printed on the packaging for further increasing visibility and consumer engagement.

Other features and aspects of the present disclosure are discussed in greater detail below.

### BRIEF DESCRIPTION OF THE DRAWINGS

A full and enabling disclosure of the present invention, including the best mode thereof to one skilled in the art, is set forth more particularly in the remainder of the specification, including reference to the accompanying figures, in which:

FIG. 1 is a perspective view of one embodiment of an absorbent article made in accordance with the present disclosure;

FIG. 2 is a plan view of the absorbent article illustrated in FIG. 1;

FIG. 3 is a plan view of one embodiment of a two dimensional code that may be used in accordance with the present disclosure;

FIG. 4 is a perspective view of another embodiment of an absorbent article made in accordance with the present disclosure;

FIG. 5 is a perspective view of another embodiment of an absorbent article made in accordance with the present disclosure;

FIG. 6 is a perspective view of a package of absorbent articles in accordance with the present disclosure;

FIG. 7 is a plan view of yet another embodiment of an absorbent article made in accordance with the present disclosure;

FIGS. 8A and 8B are plans views of another embodiment of tab members made in accordance with the present disclosure; and

FIG. 9 is a perspective view of another embodiment of an absorbent article made in accordance with the present disclosure.

Repeat use of reference characters in the present specification and drawings is intended to represent the same or analogous features or elements of the present invention.

### DETAILED DESCRIPTION

It is to be understood by one of ordinary skill in the art that the present discussion is a description of exemplary embodiments only, and is not intended as limiting the broader aspects of the present disclosure.

In general, the present disclosure is directed to an absorbent article that includes a machine readable code that is capable of conveying information to a user that is related to the product. For example, in one embodiment, the machine readable code may comprise a two dimensional code that stores information in at least two ways, such as at least in two directions. When scanned by a suitable reader device, the two dimensional code may directly provide information to a user or connect the reader device to a website address where the information is contained.

The technology discussed herein makes reference to servers, databases, software applications, and other computer-based systems, as well as actions taken and information sent to and from such systems. One of ordinary skill in the art will recognize that the inherent flexibility of computer-based systems allows for a great variety of possible configurations, combinations, and divisions of tasks and functionality between and among components. For instance, server processes discussed herein can be implemented using a single server or multiple servers working in combination. Databases and applications can be implemented on a single system or distributed across multiple systems. Distributed components can operate sequentially or in parallel. In one embodiment, for instance, the reader device used for scanning the two dimensional codes may comprise a mobile device, such as a mobile telephone that includes an optical scanner. Alternatively, the reader device may be part of or connected to a personal computer.

Thus, the various reader devices and computer systems discussed herein are not limited to any particular hardware architecture or configuration. Embodiments of the methods and systems set forth herein can be implemented by one or more general-purpose or customized reader devices and/or computing devices adapted for connecting the device to the internet. Further, when software is used in carrying out the methods of the present disclosure, any suitable programming, scripting, or other type of language or combinations of languages can be used to implement the teachings contained herein.

The machine readable codes of the present disclosure are intended to not only make information accessible to the user and convey information, but are also intended to make the absorbent articles more interactive and engaging with the consumer. In one embodiment, the machine readable code can be printed on the absorbent article. One of the challenges, however, in placing machine readable codes on absorbent articles is placing the codes on an absorbent article that is not only easily noticeable, but allows for a reader device to scan the machine readable code successfully. Thus, one concern addressed by the present disclosure is the capability of legibly placing a machine readable code on an absorbent article.

For example, one limitation with a scannable machine readable code is that the image on the absorbent article must be clear, flat and not have significant amounts of defects in order to enable the proper interaction/usability/intuitive application of the technology. For example, there are several factors that can affect the scannability of a machine readable code. These include but are not limited to print resolution, reader device capability, recognition of the machine readable code and engagement of the code by a user, and the capability of applying a machine readable code to an absorbent article having a sufficient size and at a sufficient location for scanning.

With respect to print resolution, each known printing technique for applying machine readable codes to an absorbent article will have inherent limitations in print resolution. Further, as the size of the machine readable code is changed, the print resolution may also change based on the manner in which the code is applied to the absorbent article. Further, suitable reader devices that may be used by consumers will have different specifications associated with the imaging technology on the device. Most reader devices that may be used with machine readable codes, for instance, comprise cellular telephones equipped with an optical scanner, such as a camera, in conjunction with application software. As can be appreciated, the image resolution of the camera and the software loaded into the device can change drastically from product to product. Thus, the quality of the machine readable code may become a factor in ensuring that all commercial reader devices can successfully scan the code.

Successfully overcoming the above hurdles, however, does not guarantee that the machine readable code will in fact increase the appeal of the product and/or be readily accepted by consumers. For example, in addition to the above, the machine readable code should also be highlighted on the absorbent article at a location that provides easy access and is readily noticeable by consumers.

There are many areas on an absorbent article, for instance, that may not be suitable for placement of a machine readable code in accordance with the present disclosure without altering certain aspects of the article. For instance, areas on the absorbent article where deformation occurs, such as folding, wrinkles, or other irregularities, may result in placement of a machine readable code on the article that is either not noticeable or not capable of being scanned by a suitable reader device. Such irregularities in the surfaces of the absorbent article can also change depending upon whether the article is in a flat state or whether the article has been donned by the user.

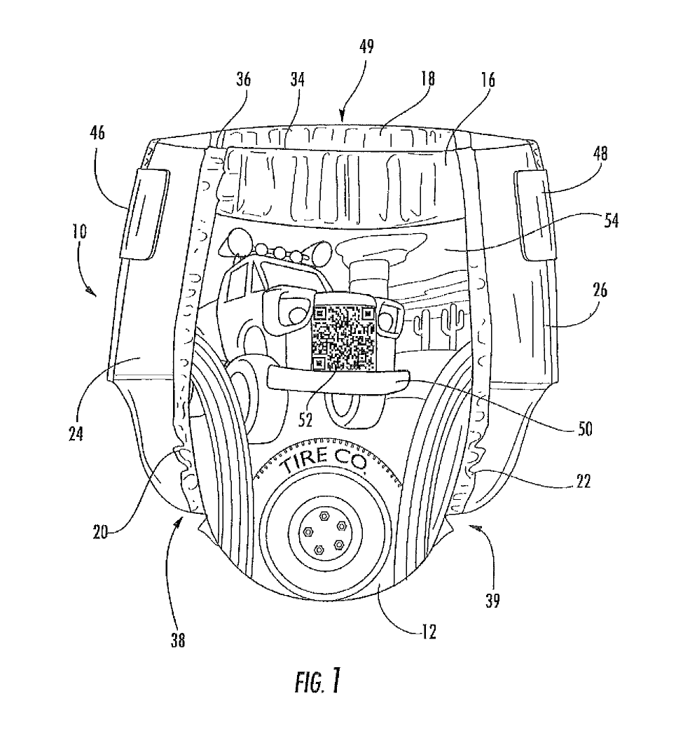

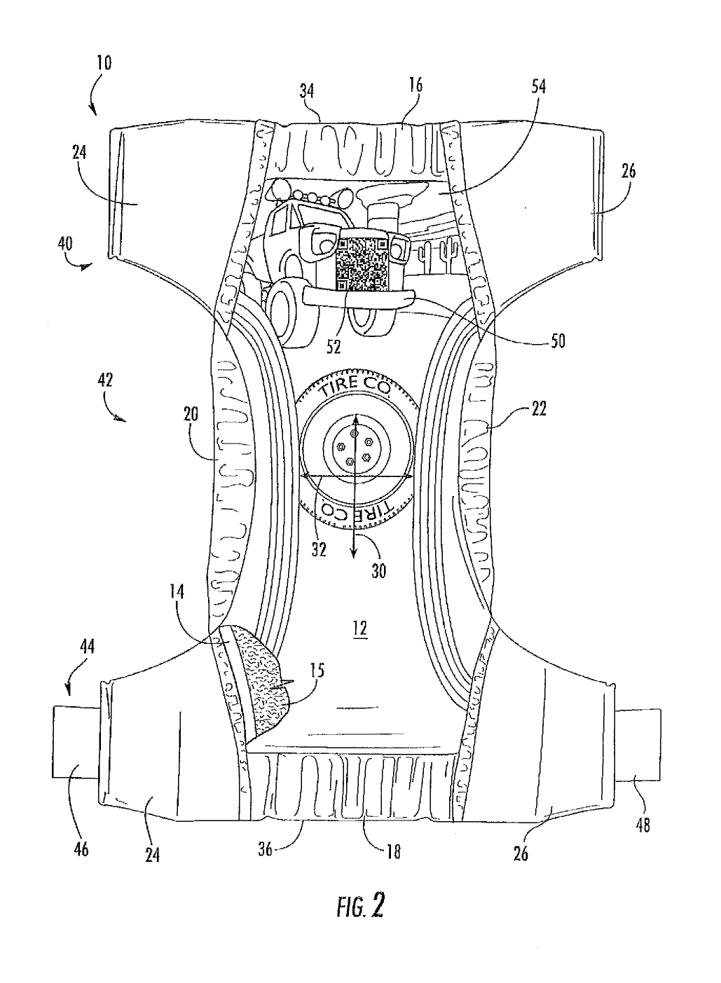

Referring to FIGS. 1 and 2, for instance, one embodiment of an absorbent article 10 made in accordance with the present disclosure is shown. In the figures, a child's training pant is generally illustrated. It should be understood, however, that the inventive concepts described herein can be applied to any suitable absorbent article, such as a diaper, an adult incontinence product, a feminine hygiene product or the like.

Referring to FIG. 2, absorbent articles generally include an outer cover 12 that includes an exterior surface and an interior surface. Located adjacent the interior surface is an absorbent structure 15. Optionally, the absorbent article can also include a liquid permeable inner lining 14. The absorbent structure can be placed in between the outer cover 12 and the inner lining 14. The absorbent article 10 can further include elastic waistband regions 16 and 18 and elastic leg members 20 and 22.

The absorbent article 10 as shown in FIG. 1 can be made from various materials. The outer cover 12 may be made from a material that is substantially liquid impermeable, and can be elastic, stretchable or nonstretchable. The outer cover 12 can be a single layer of liquid impermeable material, or may include a multi-layered laminate structure in which at least one of the layers is liquid impermeable. For instance, the outer cover 12 can include a liquid permeable outer layer and a liquid impermeable inner layer that are suitably joined together by a laminate adhesive.

For example, in one embodiment, the liquid permeable outer layer may be a spunbond polypropylene nonwoven web. The spunbond web may have, for instance, a basis weight of from about 15 gsm to about 25 gsm.

The inner layer, on the other hand, can be both liquid and vapor impermeable, or can be liquid impermeable and vapor permeable. The inner layer is suitably manufactured from a thin plastic film, although other flexible liquid impermeable materials may also be used. The inner layer prevents waste material from wetting articles such as bedsheets and clothing, as well as the wearer and caregiver. A suitable liquid impermeable film may be a polyethylene film having a thickness of about 0.2 mm.

A suitable breathable material that may be used as the inner layer is a microporous polymer film or a nonwoven fabric that has been coated or otherwise treated to impart a desired level of liquid impermeability. Other “non-breathable” elastic films that may be used as the inner layer include films made from block copolymers, such as styrene-ethylene-butylene-styrene or styrene-isoprene-styrene block copolymers.

As described above, the absorbent structure is positioned in between the outer cover and a liquid permeable bodyside liner 14. The bodyside liner 14 is suitably compliant, soft feeling, and non-irritating to the wearer's skin. The bodyside liner 14 can be manufactured from a wide variety of web materials, such as synthetic fibers, natural fibers, a combination of natural and synthetic fibers, porous foams, reticulated foams, apertured plastic films, or the like. Various woven and nonwoven fabrics can be used for the bodyside liner 14. For example, the bodyside liner can be made from a meltblown or spunbonded web of polyolefin fibers. The bodyside liner can also be a bonded-carded web composed of natural and/or synthetic fibers.

A suitable liquid permeable bodyside liner 14 is a nonwoven bicomponent web having a basis weight of about 27 gsm. The nonwoven bicomponent can be a spunbond bicomponent web, or a bonded carded bicomponent web. Suitable bicomponent staple fibers include a polyethylene/polypropylene bicomponent fiber. In this particular embodiment, the polypropylene forms the core and the polyethylene forms the sheath of the fiber. Other fiber orientations, however, are possible.

The material used to form the absorbent structure, for example, may include cellulosic fibers (e.g., wood pulp fibers), other natural fibers, synthetic fibers, woven or nonwoven sheets, scrim netting or other stabilizing structures, superabsorbent material, binder materials, surfactants, selected hydrophobic materials, pigments, lotions, odor control agents or the like, as well as combinations thereof. In a particular embodiment, the absorbent web material is a matrix of cellulosic fluff and superabsorbent hydrogel-forming particles. The cellulosic fluff may comprise a blend of wood pulp fluff. One preferred type of fluff is identified with the trade designation CR 1654, available from US Alliance Pulp Mills of Coosa, Ala., USA, and is a bleached, highly absorbent wood pulp containing primarily soft wood fibers. As a general rule, the superabsorbent material is present in the absorbent web in an amount of from about 0 to about 90 weight percent based on total weight of the web. The web may have a density within the range of about 0.1 to about 0.45 grams per cubic centimeter.

Superabsorbent materials are well known in the art and can be selected from natural, synthetic, and modified natural polymers and materials. The superabsorbent materials can be inorganic materials, such as silica gels, or organic compounds, such as crosslinked polymers. Typically, a superabsorbent material is capable of absorbing at least about 15 times its weight in liquid, and suitably is capable of absorbing more than about 25 times its weight in liquid. Suitable superabsorbent materials are readily available from various suppliers. For example, FAVOR SXM 880 superabsorbent is available from Stockhausen, Inc., of Greensboro, N.C., USA; and Drytech 2035 is available from Dow Chemical Company, of Midland, Mich., USA.

In addition to cellulosic fibers and superabsorbent materials, the absorbent pad structures may also contain adhesive elements and/or synthetic fibers that provide stabilization and attachment when appropriately activated. Additives such as adhesives may be of the same or different aspect from the cellulosic fibers; for example, such additives may be fibrous, particulate, or in liquid form; adhesives may possess either a curable or a heat-set property. Such additives can enhance the integrity of the bulk absorbent structure, and alternatively or additionally may provide adherence between facing layers of the folded structure.

The absorbent materials may be formed into a web structure by employing various conventional methods and techniques. For example, the absorbent web may be formed with a dry-forming technique, an airlaying technique, a carding technique, a meltblown or spunbond technique, a wet-forming technique, a foam-forming technique, or the like, as well as combinations thereof. Layered and/or laminated structures may also be suitable. Methods and apparatus for carrying out such techniques are well known in the art.

The absorbent web material may also be a coform material. The term “coform material” generally refers to composite materials comprising a mixture or stabilized matrix of thermoplastic fibers and a second non-thermoplastic material. As an example, coform materials may be made by a process in which at least one meltblown die head is arranged near a chute through which other materials are added to the web while it is forming. Such other materials may include, but are not limited to, fibrous organic materials such as woody or non-woody pulp such as cotton, rayon, recycled paper, pulp fluff and also superabsorbent particles or fibers, inorganic absorbent materials, treated polymeric staple fibers and the like. Any of a variety of synthetic polymers may be utilized as the melt-spun component of the coform material. For instance, in some embodiments, thermoplastic polymers can be utilized. Some examples of suitable thermoplastics that can be utilized include polyolefins, such as polyethylene, polypropylene, polybutylene and the like; polyamides; and polyesters. In one embodiment, the thermoplastic polymer is polypropylene. Some examples of such coform materials are disclosed in U.S. Pat. Nos. 4,100,324 to Anderson, et al.; 5,284,703 to Everhart, et al.; and 5,350,624 to Georger, et al.; which are incorporated herein in their entirety by reference for all purposes.

It is also contemplated that elastomeric absorbent web structures may be used. For example, an elastomeric coform absorbent structure having from about 35% to about 65% by weight of a wettable staple fiber, and greater than about 35% to about 65% by weight of an elastomeric thermoplastic fiber may be used to define absorbent pad structures according to the invention. Examples of such elastomeric coform materials are provided in U.S. Pat. No. 5,645,542, incorporated herein in its entirety for all purposes. As another example, a suitable absorbent elastic nonwoven material may include a matrix of thermoplastic elastomeric nonwoven filaments present in an amount of about 3 to less than about 20% by weight of the material, with the matrix including a plurality of absorbent fibers and a super-absorbent material each constituting about 20-77% by weight of the material. U.S. Pat. No. 6,362,389 describes such a nonwoven material and is incorporated herein by reference in its entirety for all purposes. Absorbent elastic nonwoven materials are useful in a wide variety of personal care articles where softness and conformability, as well as absorbency and elasticity, are important.

The absorbent web may also be a nonwoven web comprising synthetic fibers. The web may include additional natural fibers and/or superabsorbent material. The web may have a density in the range of about 0.1 to about 0.45 grams per cubic centimeter. The absorbent web can alternatively be a foam.

As shown in FIGS. 1 and 2, the absorbent article 10 can include side panels 24 and 26. The side panels 24 and 26 can be permanently bonded together or can be releasably attached to one another or to another region of the product. In FIG. 2, for instance, the side panels 24 and 26 are shown in an unattached state. In general, the side panels 24 and 26 are made from an elastic material, such as an elastic laminate.

As shown in FIGS. 1 and 2, each side panel may be attached to a tab member. For instance, side panel 24 is connected to a tab member 46, while side panel 26 is attached to a tab member 48. The tab members 46 and 48 may be provided on the garment to assist in donning the garment. For instance, the tab members may provide a suitable location for pulling on the garment as it is being donned. The side panels may also be used to attach the front and back of the article together so that it forms a three-dimensional configuration as illustrated in FIG. 1. In the embodiment illustrated in FIG. 1, the tab members attach to the side panels. In other embodiments, however, the tab members 46 and 48 may extend around the garment and attach to a front region of the article. In order to attach to different places on the absorbent article, the tab members may include any suitable fastening member. For instance, in one embodiment, the tab members may be coated with an adhesive for attaching to the sides or the front of the absorbent article. In another embodiment, the tab members may include hook members that engage an outside surface of the absorbent article. The hook members, for instance, can form a hook and loop attachment with materials used to form the outside surface of the article.

As shown particularly in FIG. 2, the absorbent article 10 defines a longitudinal center line 30, a transverse center line 32, a first or front longitudinal waist edge 34, and a second or back longitudinal waist edge 36. The longitudinal axis lies in the plane of the article and is generally parallel to a vertical plane that bisects a standing wearer into left and right body halves when the article is worn. The transverse axis lies in the plane of the article generally perpendicular to the longitudinal axis.

In general, the absorbent article along the longitudinal axis can be divided into a front region 40, a back region 44, and a crotch region 42 positioned in between the front region and the back region. In one embodiment, the front region, the crotch region, and the back region can all have approximately the same length in the longitudinal direction.

The absorbent article can also be divided into a front half and a back half. The front half, for instance, may extend in the longitudinal direction from the front edge to the midpoint of the product, while the back half may extend from the back edge to the midpoint of the product.

In FIG. 1, the absorbent article 10 is shown in an assembled state. In particular, the front waist edge 34 and the back waist edge 36 define a waist opening 49 which is positioned opposite two leg openings 38 and 39. When in an assembled condition, the first leg elastic member 20 at least partially surrounds the leg opening 38, while the second leg elastic member 22 at least partially surrounds the leg opening 39.

As shown in FIGS. 1 and 2, there are various areas on the absorbent article 10 that are not well suited for receiving a machine readable code in accordance with the present disclosure, unless the areas are somewhat modified. For instance, the front waistband region 16 and the back waistband region 18 typically include many wrinkles and undulations that make these locations incompatible for receiving a machine readable code, especially if the code is to be printed on the article. Thus, the waistband regions are generally not desirable for printing an image that is to be scanned and defined for user interaction.

Similarly, the areas around the leg openings 38 and 39 are also not generally suitable for receiving a printed code. Similarly, if the side panels 24 and 26 are made from an elastic laminate, these materials may also not be well suited for placement of the code.

Thus, in one embodiment, of the present disclosure, the machine readable code is applied to the absorbent article below the front and back waistband regions and generally at a position that coincides with the placement of the absorbent structure 15. In one embodiment, for instance, the machine readable code is positioned on the absorbent article below the waist opening a distance that is at least 10% of the longitudinal length of the absorbent article, such as at least 15% of the longitudinal length of the absorbent article. The machine readable code is also positioned on the absorbent article at a location that coincides with being between a first transverse side and a second transverse side of the absorbent structure 15.

Placing the machine readable code at a position that coincides with the absorbent structure 15 may provide various advantages and benefits in some applications. For instance, the absorbent structure 15 provides a structural component to the product and, in certain locations, may create a flat region of a significant size to allow for repeatable display and interaction with the scannable code.

In FIGS. 1 and 2, the absorbent article 10 generally includes various graphics on the outer cover 12 that depicts a scene. In the embodiment illustrated, for instance, the scene depicts a focal image which comprises a vehicle character 50. The character 50 appears on a background 54 with further graphics. The background 54, for instance, provides various further scene details. In the embodiment illustrated, for instance, the background 54 depicts a desert setting.

In accordance with the present disclosure, the scene further includes a machine readable code 52. As shown, the machine readable code 52 is positioned on the grill of the truck character 50. In this manner, the machine readable code 52 is not only placed on a central area of the absorbent article, but is also integrated into the scene that is depicted. Although optional, integrating the machine readable code 52 into the graphics on the absorbent article may further increase the aesthetic appeal of the product and ultimately generate more user interaction.

The machine readable code 52 as shown in FIGS. 1 and 2 generally comprises a two dimensional code that, when scanned by a suitable reader device, provides access to a user various information related to the absorbent article 10.

In the embodiment illustrated in FIGS. 1 and 2, the machine readable code is generally applied to the absorbent article through a printing process. The machine readable code can be applied to the absorbent article at the same time the scene is created on the outer cover. The machine readable code 52 may comprise a permanent graphic or an active graphic. An active graphic, for instance, is a graphic that changes when contacted with an activation agent. For instance, in one embodiment, the machine readable code 52 may comprise a disappearing graphic that disappears when contacted with an activation agent, such as a body fluid, or may comprise an appearing graphic that appears when contacted with an activation agent. Various ink compositions are available, for instance, for causing an image to appear and disappear when properly positioned on an absorbent article.

The graphic 52 illustrated in FIGS. 1 and 2 generally comprises a readable code that can convey information to a user. In one embodiment, for instance, the active graphic can comprise a two dimensional code. Two dimensional codes have evolved from simple codes, such as bar codes. Bar codes contain small amounts of information in only one direction. Although one-dimensional codes may be used in certain embodiments of the present disclosure, one-dimensional codes are very limited in the amount of information that can be conveyed to a user.

Two dimensional codes, on the other hand, can store information in at least two directions or in at least two ways, such as in a vertical and horizontal direction or in a circular direction. Two dimensional codes can also use color in addition to direction for embedding information. Various different reader devices exist that are capable of scanning two dimensional codes, such as the one illustrated in FIG. 1. In one embodiment, for instance, the reader device may include a camera that initially captures the two dimensional code in a picture and then runs the image through a decoding algorithm. Such reading devices are typically referred to as optical scanners. The algorithm allows the code to be viewed from any direction. Once decoded, the two dimensional code can either directly provide information to the reader device or link the reader device to a website address. For instance, in one embodiment, the two dimensional code may include a uniform resource locator. In one embodiment, the two dimensional code may comprise a quick response code.

Two dimensional codes can provide hard-links or physical world hyperlinks and allow people to receive information anywhere by simply scanning the code with, for instance, a camera on their cellular phone. Many cellular phones, for instance, are already equipped with reader devices or can be turned into a reader device by downloading a software application. The software is capable of deciphering a picture of the code and automatically displaying the contents back to the user.

In one embodiment, the manufacturer of the absorbent article may also provide programs and applications for downloading into mobile devices that may be focused on the particular type of machine readable code present on the product. The above may aid in the continuity of the experience and further promote interaction with the code in the product. The program or application may incorporate a feature that allows the user to access the camera of a mobile device and scan the machine readable code for redirection to a particular website for more information. The application and program downloaded to the mobile device may also provide information on how best to scan and use the machine readable codes and provide various other ideas and suggestions for their use.

Two dimensional codes can store over 7000 characters in a single image. Many codes, for instance, can encode the same amount of information into about one-tenth the amount of space of a one dimensional code.

Another advantage to using a two dimensional code as illustrated in FIG. 1 is the error correction capabilities in the event that part of the code is not readable due to damage or due to other circumstances. In such events, suitable reader devices can still decipher the image. For example, many codes have four possible levels of error correction: Level L, Level M, Level Q, and Level H which allow for 7%, 15%, 25%, and 30% of the data to be obscured yet still readable. The particular code designed for application for use with the absorbent article can have suitable data allotment for falling within one of the above levels of error correction.

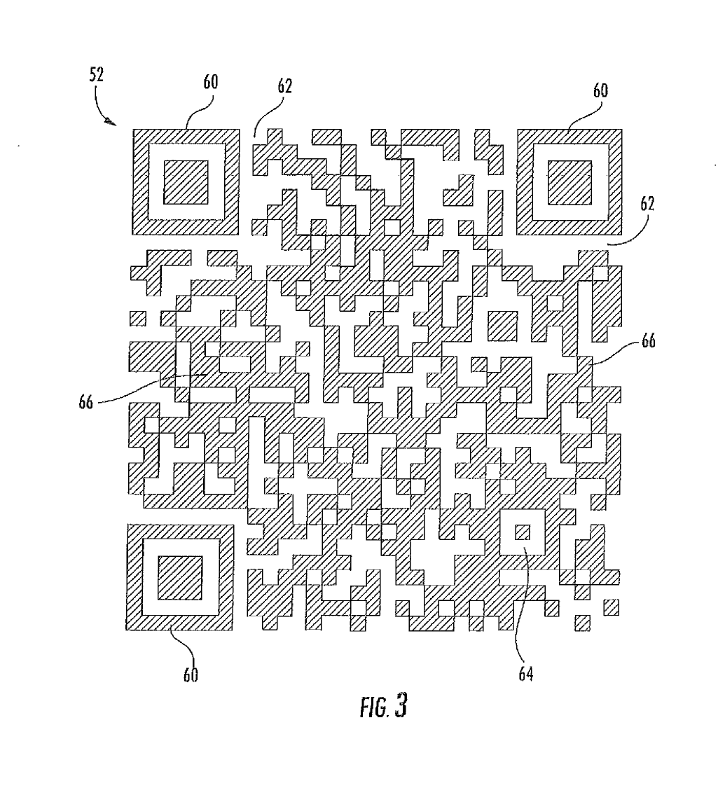

Referring to FIG. 3, an isolated view of one embodiment of a two dimensional code 52 that may be used in accordance with the present disclosure is shown. It should be understood, however, that various other two dimensional codes may be used.

As shown in FIG. 3, the two dimensional code includes various components. The different components of the code are used by the reader device to decipher the code. As described above, for instance, the reader device may be programmed with an algorithm that deciphers an image of the code.

As shown in FIG. 3, in this embodiment, the two dimensional code 52 includes a finder component 60 which, in the embodiment illustrated, includes three boxes located in three corners of the image. The finder component 60 is used by an algorithm to determine the position of the code. In certain embodiments, the elements of the finder component 60 may be surrounded by empty margins 62. The empty margins can be used to determine the dimensions of the code.

The two dimensional code 52 can further include an alignment component 64. The alignment component 64 which, in the embodiment illustrated, comprises a smaller box, may be used by an algorithm to correct skew.

In addition to the finder component 60 and the alignment component 64, the two dimensional code may further include a timing pattern which may be used as an offset marker. In one embodiment, for instance, the timing pattern may comprise a pattern that extends between adjacent finder components 60. The two dimensional code 52 further includes data component 66. In the embodiment illustrated, the data component 66 comprises the black and white modules or pixels located throughout the pattern or graphic. The different components are used by a suitable reader for recognizing the image, aligning the image, and then deciphering the data.

U.S. Pat. No. 6,786,412, U.S. Pat. No. 6,997,384, U.S. Pat. No. 5,726,435 and U.S. Patent Application Publication No. 2007/0071320, which are all incorporated herein by reference, describe and discuss various two dimensional codes such as the one illustrated in FIG. 3. In one embodiment, a suitable reader device either is programmed with an algorithm or is in communication with a programmable device that includes an algorithm for deciphering the code. Once an image of the code is scanned, the algorithm detects the position of the two dimensional code 52. Next, the code size and coefficient are determined before the cell center position is calculated. Binary data is then generated from the code that is used to reveal information directly or reveal a hyperlink.

The type of information that is conveyed by the machine readable code can vary depending upon the particular application and the desired result. In one embodiment, the machine readable code may provide information regarding a condition of the wearer. For instance, the machine readable code may provide information regarding a possible condition of the wearer and provide information not only about the condition but how to treat the condition. In an alternative embodiment, the machine readable code may provide information regarding toilet training. For instance, the information provided by the machine readable code may be for a caregiver for assisting a child in toilet training or may comprise an interactive website that encourages children to become toilet trained.

The machine readable codes can also provide information regarding various incentives. Such an incentive can include a discount or rebate for the absorbent article or for a product relating to the absorbent article. The machine readable code can also link the user to rewards, videos, and other information about the product. For example, the information conveyed to the user may discuss the benefits and advantages of the particular product, may provide product news, may discuss the launch of the next generation product, or discuss various upcoming events that are associated with the product. In one embodiment, for instance, the machine readable code may provide information not only regarding the product being used but also regarding various other collateral products that may work well in conjunction with the absorbent article. Thus, consumers can become aware of all the different products being offered by the particular manufacturer.

In yet another embodiment, the machine readable code may link a user to various surveys. The surveys, once answered by the user, can help the manufacturer improve the product or improve product service.

In the embodiment illustrated in FIGS. 1 and 2, the graphic comprising the machine readable code is integrated into the scene depicted upon the absorbent article. In particular, the machine readable code 52 is incorporated into the focal image, which, in the embodiment illustrated, comprises a character vehicle 50. Further, the machine readable code 52 is scannable from the outside surface of the absorbent article and generally is positioned in the front region of the article.

It should be understood, however, that the machine readable code may be placed at various other locations on the absorbent article. Further, the machine readable code may comprise the only graphic appearing on the absorbent article.

In one embodiment, for instance, instead of being placed on the front region of the absorbent article, the machine readable code may be applied to the back region of the absorbent article. In yet another embodiment, the machine readable code may be placed solely within the crotch region of the absorbent article. For example, when the absorbent article has been used and is disposed, certain regions of the product are typically used that are not visible or readily visible during unpacking, donning and using of the product. For example, an area that extends from the lateral center line 32 on the crotch region is typically not easily viewed when the product is being worn. After use, when the absorbent article is folded and rolled up for disposal, this region is often displayed very clearly. For example, the tab members 46 and 48 are commonly used to wrap up the product during disposal. Thus, during disposal, the crotch region on both sides of the lateral center line becomes highly visible and may, in certain applications, be an appropriate location for a machine readable code.

For example, in one embodiment, a machine readable code may be placed on the absorbent article 10 over an area that is less than about 20% of the longitudinal length of the absorbent article and centered around the lateral center line. For instance, the machine readable code may be placed within an area that extends less than about 8% of the longitudinal length of the absorbent article from each side of the lateral center line 32 in the longitudinal direction and is defined by being in between the first transverse side of the absorbent structure 15 and the second transverse side of the absorbent structure 15.

When placed in the crotch region as described above, the machine readable code 52 will be scanned often during disposable of the product. In one embodiment, once the machine readable code is scanned, information may be conveyed to the user regarding proper disposal of the product. Alternatively, the machine readable code may provide access to a user to promotions, coupons and rebates for purchasing further products.

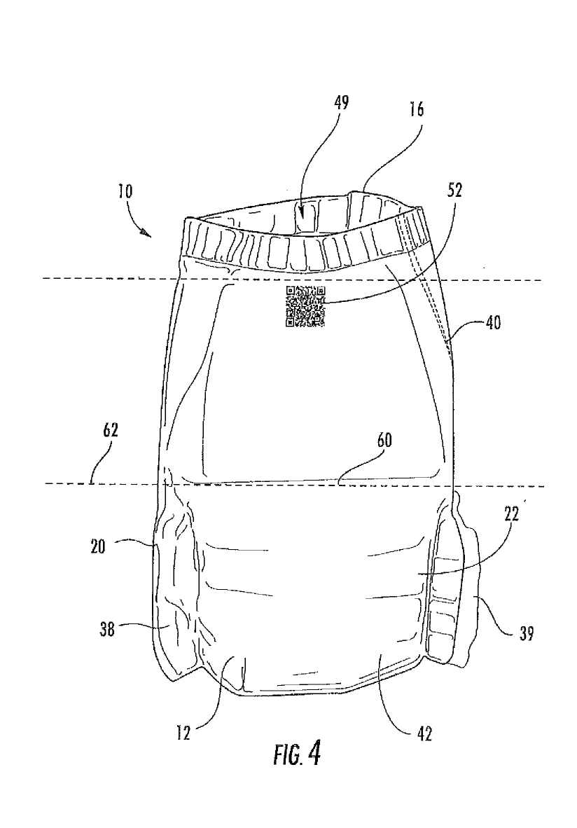

In other embodiments, the machine readable code may be placed on the absorbent article at a location that avoids places where the article has a natural tendency to form a fold or other undulations. For instance, referring to FIG. 4, another embodiment of an absorbent article 10 made in accordance with the present disclosure is shown. Like reference numerals have been used to indicate similar elements. In FIG. 4, the absorbent article 10 is shown in a folded configuration as it would normally be removed from a package. The absorbent article 10 includes a front region 40 and a crotch region 42. The back region is not shown. The absorbent article further includes a waist opening 49 opposite a first leg opening 38 and a second leg opening 39. The leg opening 38 is at least partially surrounded by a leg elastic 20. The leg opening 39, on the other hand, is partially surrounded by a second leg elastic 22.

As shown in FIG. 4, the leg elastics 22 and 20 may, in some embodiments, cause the product to bunch or form a fold 60, especially when the product is not being worn. In particular, when the leg elastics 20 and 22 are relaxed, they tend to gather the material and cause the product to possibly buckle and create valleys.

Thus, in one embodiment, the machine readable code 52 may be positioned in between a waist elastic region 16 and a transverse line 62 that is tangential to a longitudinal extent of the leg elastics 22 and 20. As shown in FIG. 4, for instance, the fold 60 typically lies along or below the transverse line 62.

When the machine readable code is scannable from an outside surface of the absorbent article, the code can be applied to the article in different ways. In one embodiment, for instance, the machine readable code can comprise a graphic that is printed on a surface of the article, such as the outside surface. The machine readable code may be printed on the article using any suitable printing process, such as inkjet printing, gravure printing, flexographic printing, and the like.

In one embodiment, the outer cover of the absorbent article may include multiple layers. The outer cover may include, for instance, an inner water impermeable film and an outer water permeable layer that may comprise, for instance, a nonwoven layer. The inner film layer may be clear such that graphics printed on a surface of the inner film layer can be visible and scannable from the outside surface of the outer cover.

In addition to being scannable from an outside surface of the absorbent article, the machine readable code may also be applied or scannable from the inside surface of the absorbent article. When scannable from the inside surface of the article, the machine readable code can be printed on the bodyside liner, a surge material, a wrap sheet that surrounds the absorbent structure, or in any other suitable layer.

As described above, the machine readable code is optimally placed at a flat location that is free from wrinkles, folds or other undulations. In one embodiment, for instance, a stiffening agent may be applied to the absorbent article in association with the machine readable code. The stiffening agent, for instance, may increase the stiffness of the surface of the absorbent article for improved scannability and readability of the code. The stiffening agent may comprise various coatings, material layers, or other constructions. In one embodiment, for instance, the stiffening agent may comprise a coating applied to a certain area on the absorbent article. The coating, for instance, may comprise an adhesive or any other suitable material capable of increasing the stiffness of either the outside surface of the absorbent article or the inside surface of the absorbent article.

In an alternative embodiment, the stiffening agent may comprise a separate piece of material that is attached to the absorbent article. The separate piece of material may comprise, for instance, a film, a woven material, a nonwoven material, or the like. For instance, in one embodiment, the stiffening agent may comprise a rigid or semi-rigid film. The machine readable code may be applied directly to the film and then attached to the absorbent article or may be placed on the absorbent article so as to coincide with the location of the stiffening agent. The machine readable code may be placed below or on top of the stiffening agent.

In yet another embodiment, the stiffening agent may comprise an embossed area on the article or may comprise a location where the absorbent structure has been de-bulked to create a flat region.

Referring to FIG. 5, another embodiment of an absorbent article in accordance with the present disclosure is shown. Like reference numerals have been used to indicate the same or similar elements. As shown, the absorbent article 10 includes a crotch region 42 positioned in between a front region 40 and a back region 44. The absorbent article further includes side panels 24 and side panels 26. In order to attach the front region 40 to the back region 44, the absorbent article further includes a first tab member 46 and a second tab member 48.

The absorbent article 10 as shown in FIG. 5 includes various elastic regions. For instance, the article includes a front waist elastic region 16 and a back waist elastic region 18. The absorbent article further includes first leg elastics 20 and second leg elastics 22. The absorbent article may also include containment flaps 70 and 72 that extend in a longitudinal direction and parallel to each other from the front region 40 to the back region 44.

In accordance with the present disclosure, the absorbent article 10 further includes a machine readable code 52. In this embodiment, the machine readable code 52 is applied to a label 53 which is then attached to the inside surface of the absorbent article within the back region 44. The label 53 can be made from any suitable material, such as a film, woven material, nonwoven material, or the like. The label 53 may also comprise a laminate containing more than one layer of material. The machine readable code 52 may be printed onto the label or otherwise applied.

When applied to a label as shown in FIG. 5, the machine readable code can be placed at almost any location on the absorbent article, even within the waistband regions.

In addition to being applied to a label as shown in FIG. 5, it should also be understood that the machine readable code 52 may be directly applied to the inside surface of the absorbent article 10 at any of the locations described above. For instance, in one embodiment, the machine readable code can be located directly on the water permeable liner at any location that corresponds with the location of the absorbent structure and is positioned in between the waistband regions 16 and 18 and the containment flaps 70 and 72.

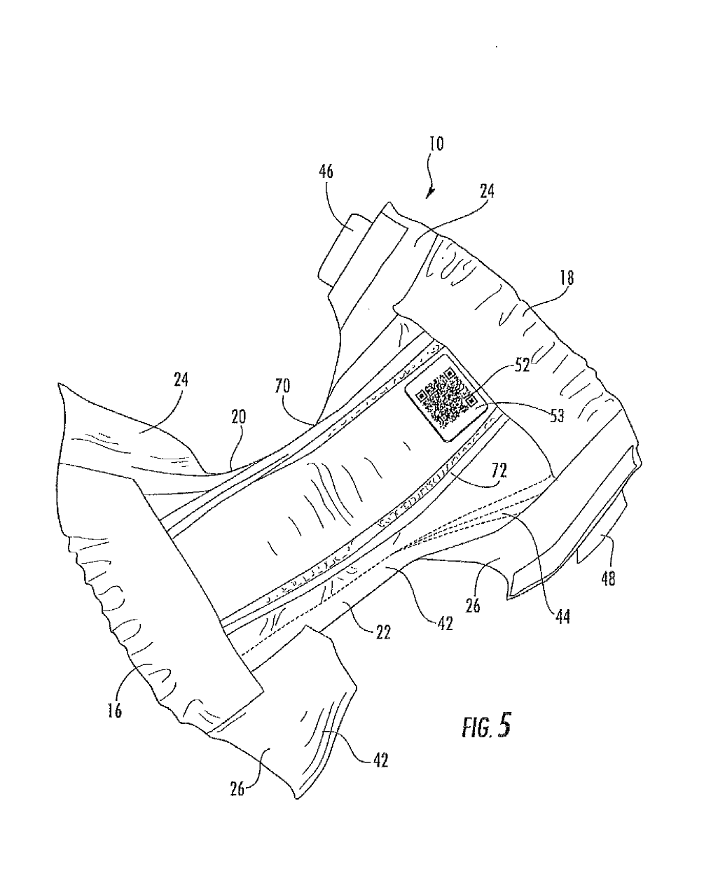

Referring to FIG. 6, a package 80 of absorbent articles 10 made in accordance with the present disclosure is shown. In this embodiment, the package 80 includes a polymer film that covers a plurality of the absorbent articles. One challenge in placing machine readable codes on absorbent articles is to present them and promote them in a way that will promote consumer interaction. In this regard, it may be desirable to have the machine readable code not only visible on the product but also on the packaging. Thus, as shown in FIG. 6, the package 80 may also display a machine readable code 84.

In addition to having the machine readable code directly on the package, as shown in FIG. 6, a machine readable code 52 on one of the absorbent articles 10 can also be located so as to be visible through a wall of the package. For instance, the package 80 may include a window or clear zone 82 through which the machine readable code 52 can be viewed and scanned. The absorbent article 10 is located in the package such that the machine readable code 52 registers with the window 82. Having the machine readable code readily noticeable from the package at the point of sale and prior to opening, further facilitates and increases consumer awareness of the code and its multiple uses.

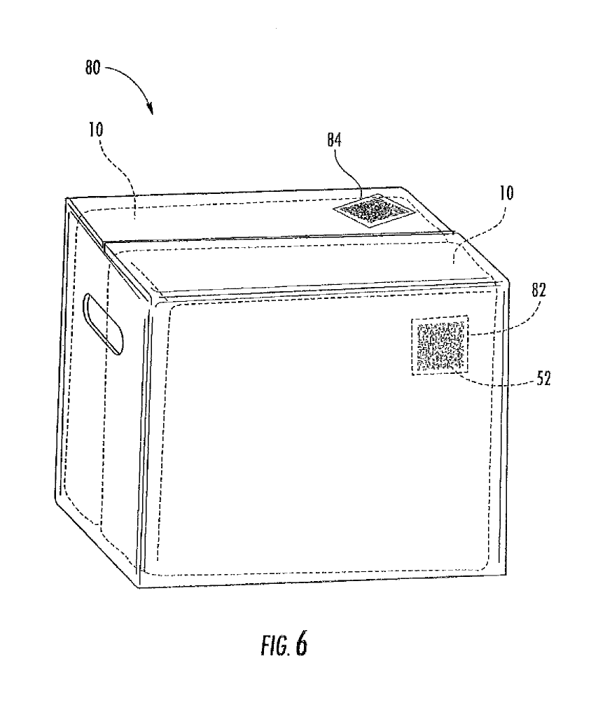

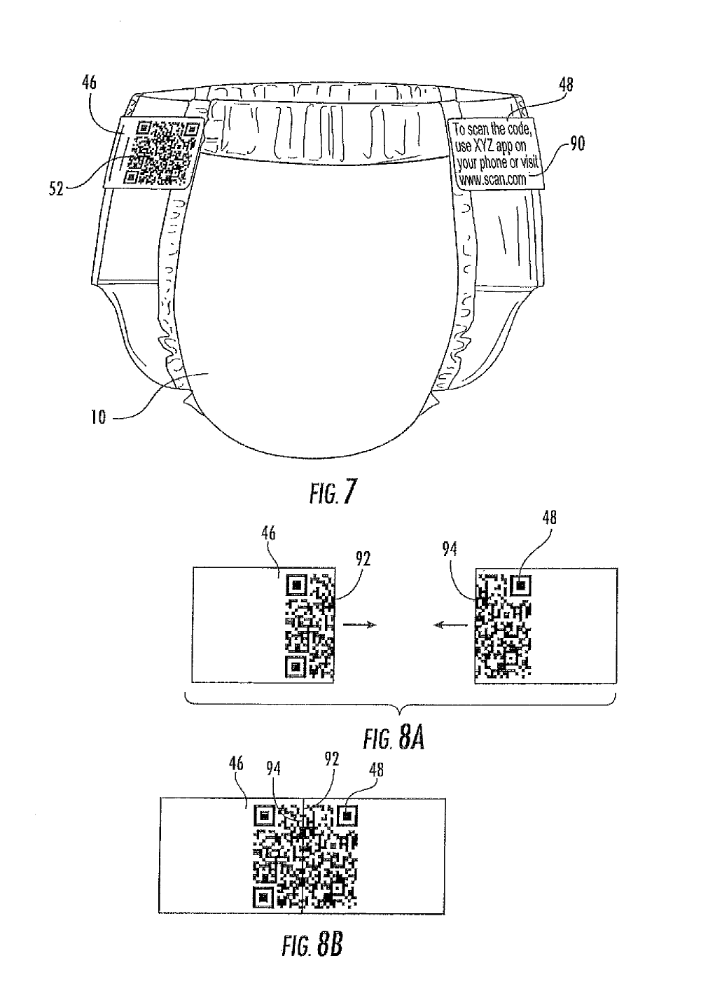

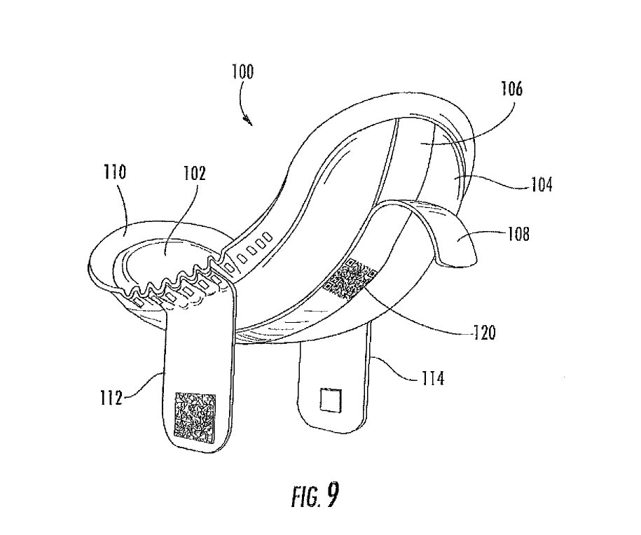

Referring to FIGS. 7, 8A and 8B, still further embodiments of absorbent articles made in accordance with the present disclosure are shown. Like reference numerals have been used to indicate similar elements.

Referring to FIG. 7, an absorbent article 10 is shown which includes tab members 46 and 48. In the embodiment illustrated, the tab members are attached to the side panels and are configured to be removably attached to the front region of the absorbent article 10. In this regard, the tab members 46 and 48 may include a fastening member, such as an adhesive or plurality of hooks for attachment to the front region of the article.

As shown in FIG. 7, in this embodiment, a machine readable code has been applied to the tab member 46. Locating the machine readable code 52 on the tab member 46 may provide various advantages and benefits. For example, tab members for use with absorbent articles are typically already made from stiff materials and thus provide a flat level surface for scanning. Further, the tab members 46 are manipulated by the user of the article and thus provide a high visibility surface for the code. In particular, the machine readable code 52 is visible during donning of the absorbent article and is also visible during disposal.

In the embodiment illustrated in FIG. 7, the opposite tab member 48 may be printed with various instructions on how to use the machine readable code 52. The tab member 48, for instance, may be printed with instructions on what type of reader devices will scan the code and how to scan the code. In the embodiment illustrated, the tab member 48 also provides a website address which can provide more detailed instructions and other information related to the machine readable code 52.

Referring to FIGS. 8A and 8B, another embodiment of tab members 46 and 48 that may be incorporated into an absorbent article in accordance with the present disclosure is shown. In this embodiment, as shown in FIGS. 8A and 8B, the first tab member 46 includes a first portion of a machine readable code 92. A second portion of the machine readable code 94 is applied to the second tab member 48.

During use of the absorbent article or during disposal, the tab members 46 and 48 may be brought together (see FIG. 8B) thus completing the machine readable code and making the code scannable. Placing portions of the code on separate tab members may increase the appeal and noticability of the code. If desired, placing portions on separate tab members may also prevent scanning of the code until the product is actually used.

In addition to diapers and training pants as shown in FIGS. 1, 2 and 4-8, the machine readable codes of the present disclosure are also well suited for use with other absorbent articles. For instance, referring to FIG. 9, a feminine hygiene product 100 is shown, particularly a sanitary napkin. The feminine hygiene product 100 includes a liquid permeable liner 102, an outer cover 104 and an absorbent structure positioned in between a liquid permeable liner and the outer cover. The absorbent structure can be a single layer of material or can be multiple layers as known in the art. The liquid permeable liner 102 and the outer cover 104 form a margin 110 that borders the absorbent structure contained within the article.

A central strip of garment adhesive 106 may extend longitudinally over the outer cover 104. A release liner 108 is shown covering the adhesive 106. When the article is being used, the release liner 108 is removed for adhering the feminine hygiene product 100 to a garment for proper positioning of the product.

Optionally, the feminine hygiene product 100 may further include a pair of garment attachment panels 112 and 114. As shown, the garment attachment panels 112 and 114 may include any suitable fastener elements, such as a mechanical fastener of conventional hook and loop type material. The fastener elements may be anchored together under the product.

In other embodiments, instead of garment attachment panels, the feminine hygiene product 100 may include lateral extending areas or the product may simply assume an oval or hourglass shape.

In accordance with the present disclosure, the feminine hygiene product 100 further includes a machine readable code 120. In the embodiment illustrated, the machine readable code 120 is printed on the release liner 108. It should be understood, however, that the machine readable code may be placed at other suitable locations. For instance, in an alternative embodiment, the machine readable code may be placed on the liquid permeable liner 102 above the absorbent structure and thus avoiding the margins 110.

In yet another embodiment, the machine readable code may be on the opposite side of the release liner 108. In this manner, once the release liner 108 is removed, the machine readable code 120 is available for scanning.

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged both in whole or in part. Furthermore, those of ordinary skill in the art will appreciate that the foregoing description is by way of example only, and is not intended to limit the invention so further described in such appended claims.

What is claimed:

1. An absorbent article comprising a crotch region positioned in between a front region and a back region, the absorbent article including a longitudinal center line and a lateral center line, the absorbent article defining an inside surface that faces a wearer and an opposite outside surface, the absorbent article including a front waist edge and a back waist edge that define a waist opening opposite two leg openings, the absorbent article having a longitudinal length that extends along the longitudinal center line from the front waist edge to the back waist edge, the absorbent article further comprising:
an outer cover having an interior surface and an exterior surface;
an absorbent structure positioned adjacent to the interior surface of the outer cover, the absorbent structure including a front longitudinal end and a back longitudinal end, the absorbent structure further including a first transverse side and a second transverse side; and
a machine readable code located on the absorbent article at a location capable of being scanned by a suitable reader device, wherein the machine readable code is configured to provide information related to the absorbent article when scanned by a suitable reader device, the machine readable code being positioned on the absorbent article below the waist opening a distance that is at least 10% of the longitudinal length of the absorbent article, the machine readable code also being positioned on the absorbent article at a location that coincides with being between the first transverse side and the second transverse side of the absorbent structure.

2. An absorbent article as defined in claim 1, wherein the machine readable code is scannable from the outside surface of the absorbent article.

3. An absorbent article as defined in claim 1, wherein the machine readable code is scannable from the inside surface of the absorbent article.

4. An absorbent article as defined in claim 1, wherein the absorbent article further includes a first leg elastic that at least partially surrounds the first leg opening and a second leg elastic that at least partially surrounds the second leg opening and wherein the machine readable code is located above a transverse line that is tangential to a longitudinal extent of the leg elastics closer to the waist opening on either the front region or the back region of the absorbent article.

5. An absorbent article as defined in claim 1, wherein the machine readable code comprises a two dimensional code.

6. An absorbent article as defined in claim 5, wherein the two dimensional code stores information in a vertical direction and in a horizontal direction.

7. An absorbent article as defined in claim 1, wherein the machine readable code stores information in a circular direction.

8. An absorbent article as defined in claim 1, wherein the machine readable code stores information in a pattern of colors.

9. An absorbent article as defined in claim 5, wherein the two dimensional code comprises a matrix pattern including a finder component and an alignment component in addition to data components.

10. An absorbent article as defined in claim 1, wherein the absorbent article further comprises graphics that depict a scene, the machine readable code comprising a graphic that is integrated into the scene.

11. An absorbent article as defined in claim 1, wherein, when scanned by a suitable reader device, the machine readable code links the reader device to a website address.

12. An absorbent article as defined in claim 11, wherein the website address provides a user with incentives, provides a user with information regarding a physiological condition, provides a user with information on product features, provides a user with information on brand related activities, provides a user with information regarding toilet training, or combinations of the above information.

13. An absorbent article as defined in claim 11, wherein the website address provides a user with incentives, the incentives comprising coupons or rebates for products.

14. An absorbent article as defined in claim 1, wherein the machine readable code is located within the crotch region.

15. An absorbent article as defined in claim 1, wherein the entire machine readable code is scannable at a location that coincides with a position of the absorbent structure within the absorbent article.

16. An absorbent article as defined in claim 1, wherein the machine readable code is scannable from an inside surface of the absorbent article, the machine readable code being located within the back region of the absorbent article.

17. An absorbent article as defined in claim 1, wherein the machine readable code is placed on the absorbent article in association with a stiffening agent.

18. An absorbent article as defined in claim 17, wherein the stiffening agent comprises a coating material applied to the absorbent article or a separate piece of material attached to the absorbent article, the absorbent article further including a liquid permeable liner, the absorbent structure being positioned in between the liquid permeable liner and the outer cover, the stiffening agent producing a location on the absorbent article that has a stiffness greater than the outer cover or the liquid permeable liner.

19. An absorbent article as defined in claim 1, wherein the machine readable code is located on a label attached to the absorbent article.

20. An absorbent article as defined in claim 1, wherein the absorbent article further includes a front elastic waist region and a back elastic waist region, the machine readable code being located below the front elastic waist region and the back elastic waist region in relation to the waist opening.

21. An absorbent article as defined in claim 1, wherein the machine readable code is printed on the absorbent article.

22. A package containing a plurality of absorbent articles as defined in claim 1, the package including a film surrounding the absorbent articles, wherein at least one machine readable code on an absorbent article is visible from an exterior of the package.

23. A package as defined in claim 22, wherein at least one machine readable code has also been applied to a surface of the package.

24. An absorbent article comprising a crotch region positioned in between a front region and a back region, the absorbent article further including a first side panel and an opposing second side panel, the side panels connecting the front region to the back region, the absorbent article further comprising:
an outer cover having an interior surface and an exterior surface;
an absorbent structure positioned adjacent to the interior surface of the outer cover;
at least one tab member located on one of the side panels; and
a machine readable code located on at least one tab member, the machine readable code being configured to provide information related to the absorbent article when scanned by a suitable reader device.

25. An absorbent article as defined in claim 24, wherein the absorbent article includes one tab member located on the first side panel and a second tab member located on the second side panel, the tab members including fastening members for attaching to the front region of the absorbent article.

26. An absorbent article as defined in claim 24, wherein the absorbent article includes a first tab member attached to the first side panel and a second tab member attached to the second side panel, a first portion of the machine readable code is located on the first tab member and a second portion of the machine readable code is located on the second tab member and wherein the machine readable code is scannable by a suitable reader device when the tab members are brought together.

27. An absorbent article as defined in claim 24, wherein the absorbent article includes a first tab member attached to the first side panel and a second tab member attached to the second side panel, the machine readable code is located on a surface of the first tab member, the second tab member including instructions for using the machine readable code.

28. A feminine hygiene or incontinence product comprising an absorbent structure positioned in between a liquid permeable liner and an outer cover, the feminine hygiene or incontinence product further comprising:
a machine readable code located on the liquid permeable liner or the outer cover at a location capable of being scanned by a suitable reader device, wherein the machine readable code is configured to provide information related to the feminine hygiene or incontinence product when scanned by a suitable reader device.

29. A feminine hygiene or incontinence product as defined in claim 28, further comprising a garment adhesive applied to the outer cover and a release liner covering the garment adhesive, the machine readable code being located on the release liner.

30. A feminine hygiene or incontinence product as defined in claim 28, wherein the machine readable code is positioned on the liquid permeable liner.
